(19) United States
(12) Patent Application Publication
(10) Pub. No.: US 2006/0020291 A1
(43) Pub. Date: Jan. 26, 2006

(54) Apparatus and method for performing nerve conduction studies with multiple neuromuscular electrodes

(76) Inventors: Shai N. Gozani, Brookline, MA (US); Mike Williams, Melrose, MA (US)

Correspondence Address: Mark J. Pandiscio;Pandiscio & Pandiscio, P.C., 470 Totten Pond Road, Waltham, MA, 02451-1914, US

(21) Appl. No.: 11/076,667

(22) Filed: Mar. 9, 2005

Related U.S. Application Data
(60) Provisional application No. 60/551,500, filed on Mar. 9, 2004.

(51) Int. Cl. A61N 1/18

(52) U.S. Cl. 607/2

(57) ABSTRACT

There is provided an apparatus for assessment of peripheral nervous system function comprising: a stimulation and data acquisition unit; at least two neuromuscular electrodes; and an adaptor unit for connecting the at least two neuromuscular electrodes with the stimulation and data acquisition unit, such that the stimulation and data acquisition unit can independently communicate with each of the neuromuscular electrodes.

### REFERENCE TO PENDING PRIOR PATENT APPLICATION

This patent application claims benefit of pending prior U.S. Provisional Patent Application Ser. No. 60/551,500, filed Mar. 9, 2004 by Shai Gozani et al. for APPARATUS AND METHOD FOR PERFORMING NERVE CONDUCTION STUDIES WITH MULTIPLE NEUROMUSCULAR ELECTRODES (Attorney Docket No. NEURO-6 PROV).

### FIELD OF THE INVENTION

This invention relates to apparatus and methods for assessment of peripheral nervous system function. More specifically, the invention relates to apparatus and methods for diagnosing peripheral nerve and muscle diseases based on assessment of neuromuscular function.

### BACKGROUND OF THE INVENTION

Peripheral Nervous System (PNS) diseases, which represent disorders of the peripheral nerves (including the spinal nerve roots) and muscles, are a common and growing health care concern. The most prevalent PNS disorders are Carpal Tunnel Syndrome (CTS), cubital tunnel syndrome low back pain caused by spinal root compression (i.e., radiculopathy) and diabetic peripheral neuropathy, which is nerve degeneration associated with diabetes. These conditions affect thirty to forty million individuals each year in the United States alone, and have an associated economic annual cost greater then $100 B. However, despite their extensive impact on individuals and the health care system, detection and monitoring of PNS diseases is based on outdated and inaccurate clinical techniques and relies on expensive referrals to specialists. In particular, effective prevention of PNS dysfunction requires early detection and subsequent action. Even experienced physicians find it difficult to diagnose and stage the severity of PNS dysfunction based on symptoms alone. The only objective way to detect many PNS diseases is to measure the transmission of neural signals. Currently, the gold standard approach is a formal nerve conduction study by a clinical neurologist, but this procedure has a number of significant disadvantages. First, a formal nerve conduction study requires a highly trained specialist. As a result, it is expensive and generally takes weeks or months to complete because of limited availability of neurologists and logistical issues such as scheduling.

Second, because they are not readily available, formal nerve conduction studies are generally performed late in the episode of care, thus serving a confirmatory role rather than a diagnostic one.

Thus, it is clear that there is a need for making accurate and robust nerve conduction measurements available to a wide variety of health care personnel in multiple settings, including the clinic, the office, the field, the workplace, etc.; collectively described as “point-of-service” settings. However, personnel in these environments may not have sufficient neurophysiological and neuroanatomical training to perform the technical elements of such studies. In particular, the correct application of nerve conduction studies requires appropriate placement of electrodes for both stimulation of the nerve and detection of the evoked response from the corresponding nerve or muscle. Furthermore, performance of a nerve conduction study requires calibration of the stimulation intensity, acquisition and measurement of evoked response waveform features, and consideration of various artifacts that can reduce the reliability of the acquired information. Therefore, in order to provide nerve conduction studies in point-of-service settings, it is necessary to simplify and automate the process of correct electrode placement and performance of the study.

The ability to perform point-of-service nerve conduction studies is substantially facilitated by the use of integrated neuromuscular electrodes, as described in U.S. Pat. No. 6,132,387. The neuromuscular electrode is an integrated device that includes stimulation and detection electrodes in a pre-configured geometry, as well as electronic features that provide information critical to the appropriate interpretation of the neurophysiological data. The neuromuscular electrode is typically placed on the patient using simple and reliable anatomical landmarks that can be readily taught to someone with minimal medical expertise. The broad utility of such neuromuscular electrodes has been demonstrated in the prior art as well as in clinical practice.

### Nerve Conduction Measurement Approaches

One particularly useful nerve conduction measurement is the conduction velocity of a nerve segment. The conduction velocity quantifies the speed, usually measured in meters per second, with which a compound nerve signal propagates between two points along the nerve. The compound nerve signal is generated by stimulating the nerve with a short electrical impulse that synchronously induces all of the participating nerve fibers to generate an action potential. A conduction velocity may be determined by stimulating a nerve at one site and recording the response at two separate sites, in which case the conduction velocity is the distance between the latter two sites divided by the propagation time between these two sites. In this case, the conduction velocity quantifies propagation between the two recording sites.

In a second, more common approach, the nerve is stimulated at two different sites, and the response recorded from a third site, separate from the two stimulation sites. In this case, the conduction velocity is defined as the distance between the two stimulation sites, divided by the propagation time between the two stimulation sites. In this case, the conduction velocity quantifies propagation between the two stimulating sites. As an example, this later configuration is used to measure the conduction velocity of the ulnar nerve across the elbow. In this situation, the ulnar nerve is stimulated both above (proximal to) and below (distal to) the elbow where the two stimulation sites are typically about 10 cm apart. The nerve response is detected as an evoked myoelectrical response from one of the ulnar innervated muscles in the hand, most commonly the Abductor Digiti Minimi (ADM) muscle.

This configuration highlights one of the fundamental challenges with performing accurate and reliable conduction velocity measurements. The stimulation and detection sites are widely distributed across the anatomy of the patient, in some configurations as much as 1 meter apart. A prior art integrated neuromuscular electrode would have to be quite large in order to accommodate the wide spacing of the stimulating and recording sites. Such a large electrode would be very costly, and might also be difficult to use. Similarly, a rigid apparatus, such as that described in U.S. Pat. Nos. 5,215,100 and 5,327,902 would be large, bulky and unlikely to effectively adapt to the wide variation found in the population.

The present invention avoids the aforementioned limitations by making it possible to use multiple, separate electrodes in nerve conduction measurements.

### SUMMARY OF THE INVENTION

In accordance with the present invention, apparatus and methods are provided for the substantially automated, rapid, and efficient assessment of PNS function without the involvement of highly trained personnel.

In one form of the present invention, there is provided an apparatus for assessment of peripheral nervous system function comprising:

a stimulation and data acquisition unit;

at least two neuromuscular electrodes; and

an adaptor unit for connecting the at least two neuromuscular electrodes with the stimulation and data acquisition unit, such that the stimulation and data acquisition unit can independently communicate with each of the neuromuscular electrodes.

In another form of the present invention, there is provided an apparatus for assessment of peripheral nervous system function comprising:

at least two neuromuscular electrodes for stimulating a nerve and/or detecting a bioelectrical signal;

a stimulation and data acquisition unit comprising:
a stimulator for generating electrical pulses to stimulate a nerve in a patient;
a data acquisition component for acquiring a bioelectrical signal from a patient; and

an adaptor unit for connecting the at least two neuromuscular electrodes with the stimulation and data acquisition unit such that the stimulation and data acquisition unit can independently communicate with each of the neuromuscular electrodes.

In another form of the present invention, there is provided a method for assessment of peripheral nervous system function comprising:

providing:
a stimulation and data acquisition unit;
at least two neuromuscular electrodes; and
an adaptor unit for connecting the at least two neuromuscular electrodes with the stimulation and data acquisition unit, such that the stimulation and data acquisition unit can independently communicate with each of the neuromuscular electrodes;

connecting the at least two neuromuscular electrodes with the stimulation and data acquisition unit using the adapter unit, such that the stimulation and data acquisition unit can independently communicate with each of the neuromuscular electrodes;

independently calculating for at least one electrode the lowest possible stimulation level of electrical impulses that provides analyzable signals;

applying the stimulation signal to a nerve; and

detecting an anatomical response.

### BRIEF DESCRIPTION OF THE DRAWINGS

These and other objects, features and attendant advantages of the present invention will become apparent from a consideration of the following detailed description of the preferred embodiment of the invention, which are to be considered in conjunction with the accompanying drawings wherein like numbers refer to like parts and further wherein:

FIG. 1 is a schematic diagram of the system for performing nerve conduction studies in accordance the present invention;

FIG. 2 is a schematic diagram of a neuromuscular electrode (B₁) that contains both stimulation and detection sites;

FIG. 3 is a schematic diagram of another type of neuromuscular electrode (B₂) that contains no detector sites but two sets of stimulation for nerve stimulation electrodes at two different anatomical locations along the course of the nerve; and

FIG. 4 is a schematic diagram of a stimulation and data acquisition unit (SDAU) in accordance with the present invention.

### DETAILED DESCRIPTION OF THE PREFERRED EMBODIMENTS

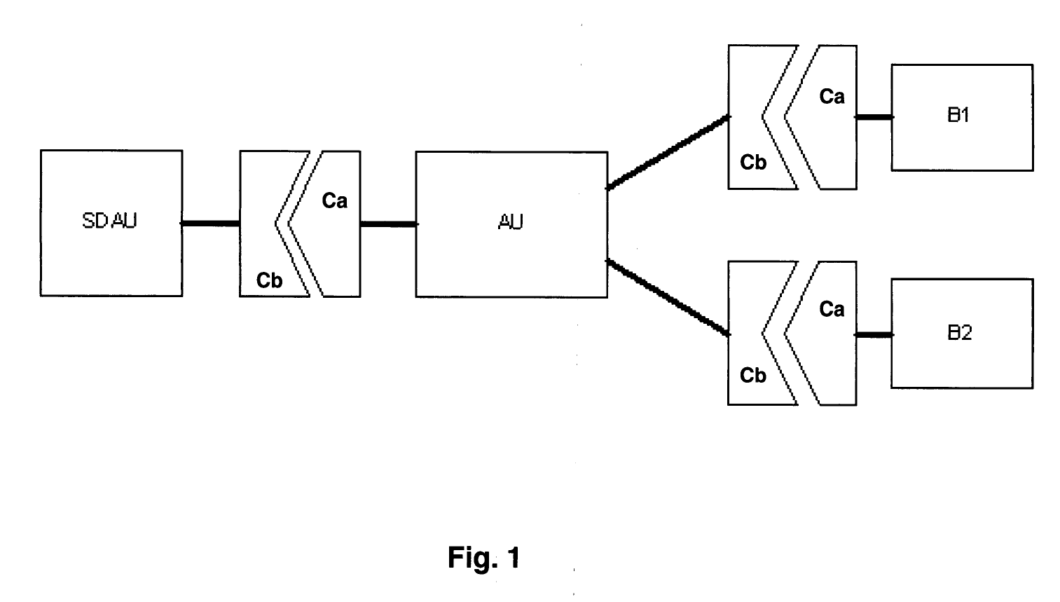

In one embodiment of the present invention, and looking now at FIG. 1, the apparatus for performing nerve conduction studies comprises a Stimulation and Data Acquisition Unit SDAU, an adaptor unit AU, and two or more neuromuscular electrodes B₁, B₂. The adaptor unit AU provides a “smart” connection between the neuromuscular electrodes B₁, B₂ and the stimulation and data acquisition unit SDAU.

### Neuromuscular Electrodes B₁, B₂

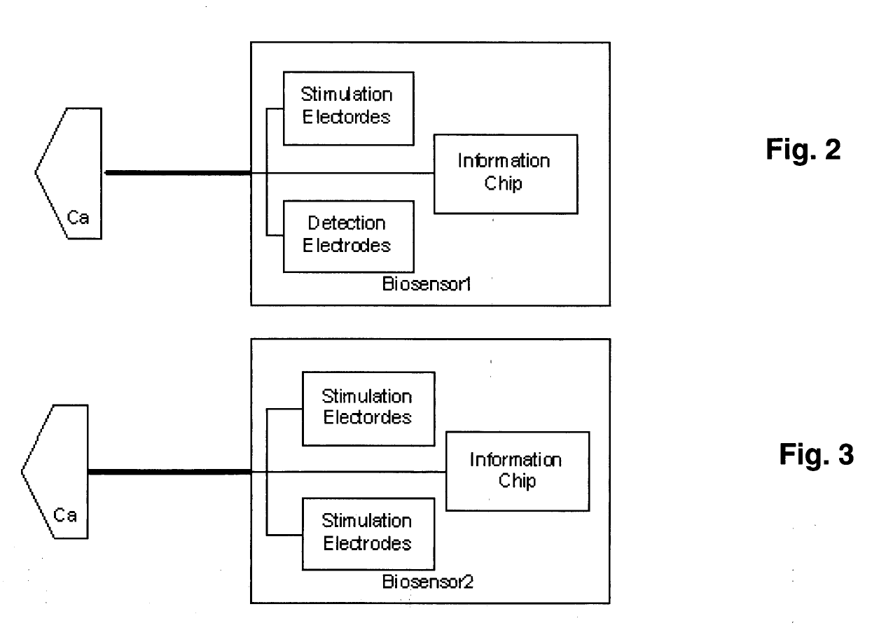

Neuromuscular electrodes B₁, B₂ (see FIGS. 2 and 3) contain stimulation sites for nerve stimulation and bioelectrical signal detection in an integrated package. The biosensor of neuromuscular electrodes B₁, B₂ may comprise one or more flexible layers. In addition, a layer of medical grade adhesive may be provided so as to ensure that once the biosensor is placed on the skin, it remains in place. The electrodes are preferably constructed by cutting out wells in the adhesive and filling them with conducting gel. One or more flexible layers of electrical traces are used to transmit the electrical signals to and from the electrodes. The traces are routed to a connector on the biosensor. A top layer may contain graphics for guiding the user in placement of the biosensor. The biosensor is designed so that if its perimeter is properly aligned with easily identifiable anatomical landmarks, the electrodes will automatically be located over proper stimulation and detection points. The biosensor may also contain a chip with nonvolatile memory. At the time of manufacturing, the chip is encoded with information specifying the type of sensor. The biosensor may also contain a temperature probe that measures skin surface temperature and electronically transmits the information to the SDAU. In a preferred embodiment, the chip encoded with sensor type information and the temperature probe are integrated into the same microchip package. The biosensors are designed for one-time use. Designing the biosensors as disposable components is primarily dictated by the issues with cross patient contamination. Also, from a functional point of view, signal quality is seriously compromised with multiple uses of the electrode gel. The traces that route signals to and from the biosensor electrodes are routed through a connector.

The neuromuscular electrode (B₁) contains both stimulation and detection sites.

The neuromuscular electrode (B₂) contains no detector sites but two sets of stimulation for nerve stimulation electrodes at two different anatomical locations along the course of the nerve. Another embodiment of a neuromuscular electrode contains one or more detection sites but no stimulation sites.

### Stimulation and Data Acquisition Unit SDAU

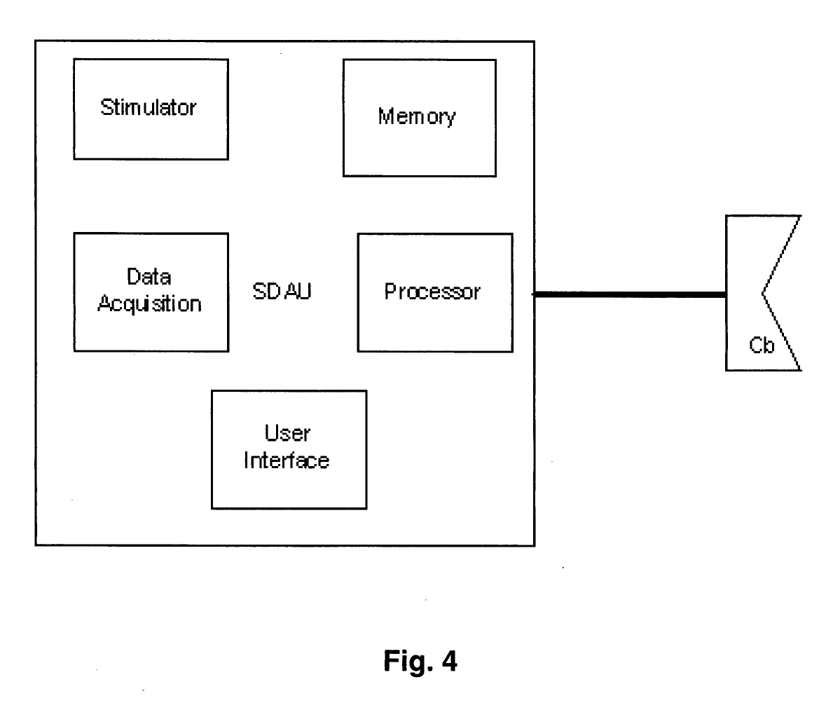

FIG. 4 illustrates a block diagram of an embodiment of a Stimulation and Data Acquisition Unit SDAU. SDAU contains a number of components working in concert. The first component is a stimulator for generating electrical pulses of varying duration and magnitude sufficient to stimulate different nerves across a very high percentage of the patient population. Stimulation levels necessary to provide sufficient nerve response vary from patient to patient and from nerve to nerve. If stimulation levels are too low, the response signals are not of sufficient amplitude or quality to perform diagnostic assessments. Conversely, if stimulations are higher than necessary, patient comfort is compromised, so it is not practical to simply apply a large stimulus across the patient population. Neurologists typically manually adjust stimulation levels to find the window of sufficient response signals with the lowest possible stimulation.

In one embodiment of the present invention, there is provided a search algorithm, referred to as Stimulus Gain Control (SGC). This algorithm is used to find the lowest possible stimulation level for each stimulation electrode that provides analyzable signals. The SGC search algorithm is carried out at the start of the test. Once the proper stimulation level is determined by the SGC search algorithm, the diagnostic stimulations are carried at that level.

The Data Acquisition component of the SDAU controls the signal acquisition process from the biosensor detection electrodes. In one embodiment of the present invention, a primary task is to control the start of data acquisition with respect to the time of stimulation and the size of the data acquisition window. Once the signal is acquired, it is sent through various gain and filter stages, and then it is converted from analog signal to digital signal. Certain settings for this process are fixed by the hardware capabilities. Other settings for this process are adjusted on a patient, nerve or stimulation site basis. For example, in stimulating the same nerve at two different locations, the time of arrival between the stimulation electrodes and common detection electrodes is different.

The memory component of the SDAU is used to store the waveforms for further processing.

In addition, the SDAU unit contains one or more processors for signal processing, control of the stimulation and data acquisition process, and control of the unit's hardware. A user interface accepts input from the clinical user and displays test results.

### Adaptor Unit AU

In a preferred embodiment of the present invention, there is provided an Adaptor Unit AU which serves as a “smart” interface between the SDAU unit and the neuromuscular electrodes B₁, B₂ such that the SDAU can independently communicate with each of the electrodes B₁, B_{2,}, e.g., during calibration and nerve testing. The Adaptor Unit is a connector with well-defined stimulation, detection and communication lines between the SDAU unit and the neuromuscular electrodes B₁, B₂. The simplest test uses a neuromuscular electrode containing both stimulation and detection sites. The neuromuscular electrode is connected directly to the SDAU and a single test is performed. A test refers to a series of stimulations and recordings of the resultant signals from a particular stimulation/detection pair.

Advantages of the Present Invention

As described above, in certain nerve conduction studies, the stimulation and detection sites are at a significant distance from each other. Other nerve conduction studies require multiple stimulation sites. A single, large neuromuscular electrode is currently possible to use for such nerve conduction studies, but it is unfeasible for a number of reasons. The cost of a neuromuscular electrode is directly related to its size. The cost of a large neuromuscular electrode becomes prohibitive especially for a disposable component. Also, patient application becomes difficult with large neuromuscular electrodes. Hence, multiple small neuromuscular electrodes are desirable. For tests requiring multiple electrodes, another component—the Adapter Unit—is required.

The present invention provides such Adapter Unit AU. As shown in FIG. 3, Adaptor Unit AU contains a connector Ca, equivalent to that found on the neuromuscular electrode. The SDAU interfaces to the AU through connector Cb. The AU contains two or more additional connectors Cb, equivalent to the SDAU connector, for interfacing with two or more neuromuscular electrodes B₁, B₂. In this way, the AU allows for simultaneous mechanical connection between the SDAU and multiple neuromuscular electrodes B₁, B₂. Internally, the adaptor unit AU contains switching mechanisms that connect the electrical connections between the SDAU and selected detector and stimulator lines on the individual neuromuscular electrodes B₁, B₂. In use, the clinician anatomically places the necessary neuromuscular electrodes for the particular nerve test and connects them to the AU connectors. Particular neuromuscular electrodes need to be connected to specific cables on the AU. The SDAU is also connected to the AU and the test is initiated. The software on the SDAU polls the neuromuscular electrodes and reads each type. The software checks if the neuromuscular electrodes and the AU cables they are connected to form a valid configuration. If so, the test is allowed to commence with stimulations delivered. If the neuromuscular electrode and their connections to the AU do not form a valid configuration, the user is informed of the status and the test is halted with no stimulations delivered.

For each valid neuromuscular electrode/AU connection configuration, the SDAU control software contains a protocol for setting the internal AU switches. This allows stimulation and detection at the appropriate anatomical location throughout a particular nerve test. The SDAU performs a test with a particular set of stimulation locations. The data is marked as coming from this configuration and processed. The SDAU then commands the AU switches to another set of stimulator and/or detector locations and the process repeats itself. This highly automates the process of selecting stimulus and detector locations and processing the data for each test appropriately.

### Modifications

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. Apparatus for assessment of peripheral nervous system function comprising:
a stimulation and data acquisition unit;
at least two neuromuscular electrodes; and
an adaptor unit for connecting the at least two neuromuscular electrodes with the stimulation and data acquisition unit, such that the stimulation and data acquisition unit can independently communicate with each of the neuromuscular electrodes.

2. Apparatus for assessment of peripheral nervous system function comprising:
at least two neuromuscular electrodes for stimulating a nerve and/or detecting a bioelectrical signal;
a stimulation and data acquisition unit comprising:
a stimulator for generating electrical pulses to stimulate a nerve in a patient;
a data acquisition component for acquiring a bioelectrical signal from a patient; and
an adaptor unit for connecting the at least two neuromuscular electrodes with the stimulation and data acquisition unit such that the stimulation and data acquisition unit can independently communicate with each of the neuromuscular electrodes.

3. Apparatus according to claim 2 wherein the stimulator produces stimulation levels necessary to provide sufficient nerve response in a patient.

4. Apparatus according to claim 3 wherein the stimulator produces stimulation levels calculated by a search algorithm adapted to calculate the lowest possible stimulation level of electrical impulses that provides analyzable signals.

5. Apparatus according to claim 2 wherein the top layer of the one or more electrodes contains graphics for guiding the user in placement of the biosensor.

6. Apparatus according to claim 2 wherein the electrode is designed so that if its perimeter is properly aligned with easily identifiable anatomical landmarks, the electrode is automatically located over proper stimulation and detection points.

7. Apparatus according to claim 2 wherein each of the neuromuscular electrodes comprises a connector of a first type, the stimulation and data acquisition unit comprises a counterpart connector of the second type; and further wherein the adaptor unit comprises a connector of the first type, and at least two connectors of the second type.

8. A method for assessment of peripheral nervous system function comprising:
providing:
a stimulation and data acquisition unit;
at least two neuromuscular electrodes; and
an adaptor unit for connecting the at least two neuromuscular electrodes with the stimulation and data acquisition unit, such that the stimulation and data acquisition unit can independently communicate with each of the neuromuscular electrodes;
connecting the at least two neuromuscular electrodes with the stimulation and data acquisition unit using the adapter unit, such that the stimulation and data acquisition unit can independently communicate with each of the neuromuscular electrodes;
independently calculating for at least one electrode the lowest possible stimulation level of electrical impulses that provides analyzable signals;
applying the stimulation signal to a nerve; and
detecting an anatomical response.
